(19) United States
(12) Patent Application Publication
(10) Pub. No.: US 2026/0162761 A1
(43) Pub. Date: Jun. 11, 2026

(54) TECHNIQUES FOR GENERATING MOLECULES WITH FRAGMENT RETRIEVAL AUGMENTATION

(71) Applicant: NVIDIA CORPORATION, Santa Clara, CA (US)

(72) Inventors: Weili NIE, Sunnyvale, CA (US); Karsten KREIS, Vancouver (CA); Seul LEE, Santa Clara, CA (US); Meng LIU, San Jose, CA (US); Saee PALIWAL, Brooklyn, NY (US); Srimukh Prasad VECCHAM KRISHNA PRASAD, Menlo Park, CA (US); Daniel Alexander REIDENBACH, San Jose, CA (US); Arash VAHDAT, San Mateo, CA (US)

(21) Appl. No.: 19/080,611

(22) Filed: Mar. 14, 2025

Related U.S. Application Data
(60) Provisional application No. 63/657,712, filed on Jun. 7, 2024.
(60) Provisional application No. 63/658,186, filed on Jun. 10, 2024.

(51) Int. Cl. G16B 15/00; G16B 40/20; G16C 20/50; G16C 20/70

(52) U.S. Cl. CPC G16B 15/00; G16B 40/20; G16C 20/50; G16C 20/70

(57) ABSTRACT

The disclosed method for generating molecules includes selecting, based on one or more molecule properties, one or more hard molecule fragments and one or more soft molecule fragments; and processing, using a trained machine learning model, the one or more hard molecule fragments and the one or more soft molecule fragments to generate a molecule, where the molecule includes the one or more hard molecule fragments, and the trained machine learning model generates the molecule based on the one or more soft molecule fragments.

### CROSS-REFERENCE TO RELATED APPLICATIONS

This application claims priority benefit of the United States Provisional Patent Application titled, “MOLECULE GENERATION WITH FRAGMENT RETRIEVAL AUGMENTATION,” filed on Jun. 7, 2024, and having Ser. No. 63/657,712 and United States Provisional Patent Application titled, “MOLECULE GENERATION WITH FRAGMENT RETRIEVAL AUGMENTATION,” filed on Jun. 10, 2024, and having Ser. No. 63/658,186. The subject matter of these related applications is hereby incorporated herein by reference.

### BACKGROUND

### Technical Field

Embodiments of the present disclosure relate generally to computer science, artificial intelligence, and machine learning, and more specifically, to techniques for generating molecules with fragment retrieval augmentation.

### Description of the Related Art

The discovery and development of new molecules is crucial to many scientific and industrial fields. For example, in drug discovery, new molecules can be used to bind specific biological targets to treat associated diseases, while reducing side effects. As another example, in materials science, new molecules can be used in advanced polymers, nanomaterials, and catalysts with enhanced performance characteristics. As a further example, in the energy sector, new molecules can be used in battery components, fuel cell materials, and solar energy absorbers.

One conventional approach for discovering and optimizing new molecules with desired properties is through experimentation. Such experimentation typically relies on trial and error to test different molecules. However, testing different molecules through trial and error is oftentimes very time consuming and labor intensive. Further, some molecules having the desired properties may not be tested, which can result in the most suitable molecules being overlooked during trial and error testing.

To avoid experimentation, automated approaches have been developed to generate new molecules using computers. One conventional approach for generating a molecule that has desired properties is to combine known molecule fragments having those properties into a new molecule. Each known molecule fragment is a small, defined portion of a known molecule that represents a structural unit or substructure within the known molecule. Multiple known molecule fragments and properties associated with those fragments can be stored in a database. Given a set of desired properties, the database can be searched to identify molecule fragments that best satisfy those properties. The identified molecule fragments can then be combined into a new molecule.

One drawback of the above approach for generating molecules is the generated molecules are limited to combinations of known molecule fragments. In some cases, the known molecule fragments may not be combinable into molecules that exhibit desired properties. For example, the set of properties could include high binding affinity to a particular protein. However, if none of the known molecule fragments have such a high binding affinity, then combinations of the known molecule fragments may also lack high binding affinity to the particular protein. Because the above approach cannot improve beyond what is achievable by combining the known molecule fragments, molecules having desired properties cannot be generated in many cases.

As the foregoing illustrates, what is needed in the art are more effective techniques for generating molecules.

### SUMMARY

One embodiment of the present disclosure sets forth a computer-implemented method for generating molecules. The method includes selecting, based on one or more molecule properties, one or more hard molecule fragments and one or more soft molecule fragments. The method further includes processing, using a trained machine learning model, the one or more hard molecule fragments and the one or more soft molecule fragments to generate a molecule. The molecule includes the one or more hard molecule fragments, and the trained machine learning model generates the molecule based on the one or more soft molecule fragments.

Another embodiment of the present disclosure sets forth a computer-implemented method for training a machine learning model to generate molecules. The method includes selecting a plurality of molecule fragments that are most similar to a first molecule fragment included in a first molecule. The method further includes processing, using an untrained machine learning model, one or more other molecule fragments included in the first molecule, the first molecule fragment, and the plurality of molecule fragments except for a second molecule fragment included in the plurality of molecule fragments to generate a second molecule. In addition, the method includes updating, based on a comparison between a third molecule fragment included in the second molecule and the second molecule fragment, one or more parameters of the untrained machine learning model to generate a trained machine learning model.

Other embodiments of the present disclosure include, without limitation, one or more computer-readable media including instructions for performing one or more aspects of the disclosed techniques as well as one or more computing systems for performing one or more aspects of the disclosed techniques.

At least one technical advantage of the disclosed techniques relative to the prior art is that, with the disclosed techniques, molecules are generated that include, but are not limited to, known molecule fragments. In some cases, the generated molecules can exhibit a set of desired properties to a higher degree than molecules that are generated by simply combining known molecule fragments. That is, a broader range of molecules can be generated using the disclosed techniques, increasing the likelihood of generating molecules with improved properties over prior art approaches. Further, molecules that are generated according to the disclosed techniques can generally be synthesized in real life. These technical advantages represent one or more technological improvements over prior art approaches.

### BRIEF DESCRIPTION OF THE DRAWINGS

So that the manner in which the above recited features of the various embodiments can be understood in detail, a more particular description of the inventive concepts, briefly summarized above, may be had by reference to various embodiments, some of which are illustrated in the appended drawings. It is to be noted, however, that the appended drawings illustrate only typical embodiments of the inventive concepts and are therefore not to be considered limiting of scope in any way, and that there are other equally effective embodiments.

FIG. 1 illustrates a block diagram of a computer-based system configured to implement one or more aspects of the various embodiments;

FIG. 2 is a more detailed illustration of the machine learning server of FIG. 1, according to various embodiments;

FIG. 3 is a more detailed illustration of the computing device of FIG. 1, according to various embodiments;

FIG. 4 is a more detailed illustration of the molecule generating application of FIG. 1, according to various embodiments;

FIG. 5 is a more detailed illustration of the model trainer of FIG. 1, according to various embodiments;

FIG. 6 is a flow diagram of method steps for training a molecular generative model, according to various embodiments; and

FIG. 7 is a flow diagram of method steps for generating molecules using a trained molecular generative model, according to various embodiments.

### DETAILED DESCRIPTION

In the following description, numerous specific details are set forth to provide a more thorough understanding of the various embodiments. However, it will be apparent to one skilled in the art that the inventive concepts may be practiced without one or more of these specific details.

### General Overview

Embodiments of the present disclosure provide techniques for generating molecules using fragment retrieval augmentation. In some embodiments, a molecule generating application takes as input desired properties of a molecule. The molecule generating application retrieves, from a fragment vocabulary, a number of hard fragments that a newly generated molecule must include and a number of soft fragments that guide the generation of the new molecule. It should be noted that, as used herein, generating a molecule refers to generating the design of a molecule rather than manufacturing a physical molecule. The molecule generating application processes the hard fragments and the soft fragments using a trained molecular generative model to generate a new molecule. The molecule generating application adds the new molecule to a molecule population. The molecule generating application also decomposes the new molecule into new molecule fragments that are added to the fragment vocabulary. Optionally, the molecule generating application performs genetic modification, such as crossover and mutation operations, using molecules in the molecule population to generate modified molecules, which can be added to the molecule population and decomposed into molecule fragments that are added to the fragment vocabulary. The foregoing process can be repeated any number of times to generate molecules and fragments that increasingly satisfy the desired molecule properties received as input.

To train the molecular generative model, a model trainer uses a number of molecules from a training dataset. For each molecule selected from the training dataset, the model trainer retrieves multiple fragments that are most similar to a first fragment in the selected molecule. The model trainer inputs (1) other fragments in the selected molecule as hard fragments, and (2) the first fragment and the multiple other fragments that are most similar to the first fragment, except for a most similar fragment to the first fragment, into the molecular generative model being trained. Given such inputs, the molecular generative model outputs a new molecule. Then, the model trainer updates parameters of the molecular generative model based on a comparison, such as a cross-entropy loss, between a fragment in the new molecule corresponding to the first fragment and the most similar fragment to the first fragment.

The techniques for generating molecules have many real-world applications. For example, those techniques could be applied to generate molecules that are useful in drug discovery and development, material science, chemical research, agrochemicals, cosmetics, batteries, and industrial applications, among other things.

The above examples are not in any way intended to be limiting. As persons skilled in the art will appreciate, as a general matter, the techniques for generating molecules can be implemented in any suitable application.

### System Overview

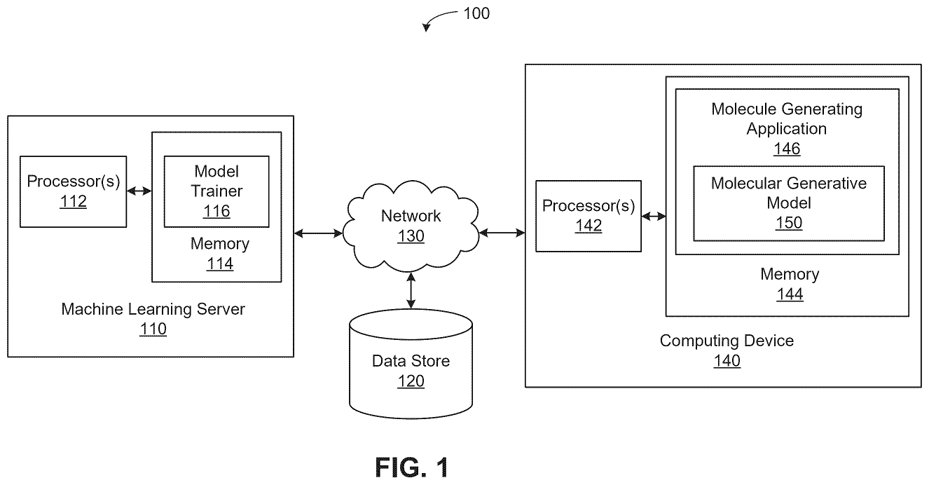

FIG. 1 illustrates a block diagram of a computer-based system 100 configured to implement one or more aspects of at least one embodiment. As shown, the system 100 includes, without limitation, a machine learning server 110, a data store 120, and a computing device 140 in communication over a network 130, which can be a wide area network (WAN) such as the Internet, a local area network (LAN), a cellular network, and/or any other suitable network. The machine learning server 110 includes, without limitation, one or more processors 112 and a system memory 114. The system memory 114 stores, without limitation, a model trainer 116. The computing device 140 includes, without limitation, one or more processors 142 and a system memory 144. The system memory 144 stores, without limitation, a molecule generating application 146 that includes a molecular generative model 150.

As shown, the model trainer 116 executes on the processor(s) 112 of the machine learning server 110 and is stored in the system memory 114 of the machine learning server 110. The processor(s) 112 receive user input from input devices, such as a keyboard or a mouse. In operation, the one or more processors 112 may include one or more primary processors of the machine learning server 110, controlling and coordinating operations of other system components. In particular, the processor(s) 112 can issue commands that control the operation of one or more graphics processing units (GPUs) (not shown) and/or other parallel processing circuitry (e.g., parallel processing units, deep learning accelerators, etc.) that incorporates circuitry optimized for graphics and video processing, including, for example, video output circuitry. The GPU(s) can deliver pixels to a display device that can be any conventional cathode ray tube, liquid crystal display, light-emitting diode display, and/or the like.

The system memory 114 of the machine learning server 110 stores content, such as software applications and data, for use by the processor(s) 112 and the GPU(s) and/or other processing units. The system memory 114 can be any type of memory capable of storing data and software applications, such as a random-access memory (RAM), a read-only memory (ROM), an erasable programmable read-only memory (EPROM or Flash ROM), or any suitable combination of the foregoing. In some embodiments, a storage (not shown) can supplement or replace the system memory 114. The storage can include any number and type of external memories that are accessible to the processor 112 and/or the GPU. For example, and without limitation, the storage can include a Secure Digital Card, an external Flash memory, a portable compact disc read-only memory (CD-ROM), an optical storage device, a magnetic storage device, and/or any suitable combination of the foregoing.

The machine learning server 110 shown herein is for illustrative purposes only, and variations and modifications are possible without departing from the scope of the present disclosure. For example, the number of processors 112, the number of GPUs and/or other processing unit types, the number of system memories 114, and/or the number of applications included in the system memory 114 can be modified as desired. Further, the connection topology between the various units in FIG. 1 can be modified as desired. In some embodiments, any combination of the processor(s) 112, the system memory 114, and/or GPU(s) can be included in and/or replaced with any type of virtual computing system, distributed computing system, and/or cloud computing environment, such as a public, private, or a hybrid cloud system.

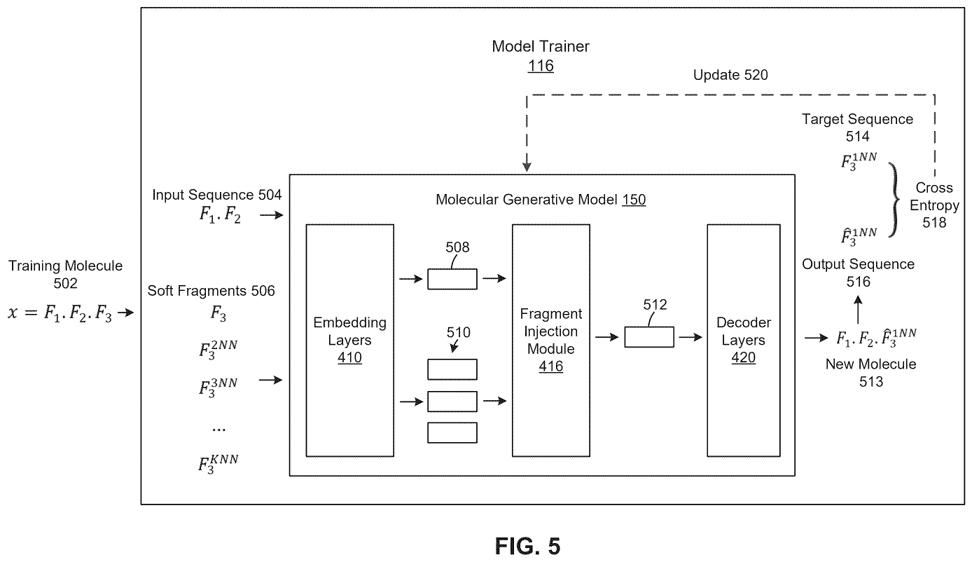

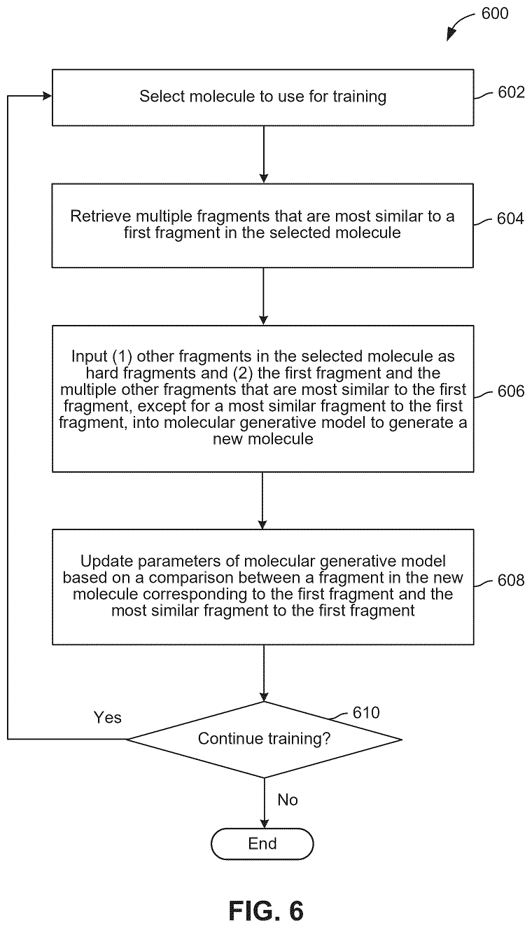

In some embodiments, the model trainer 116 is configured to train one or more machine learning models, including a molecular generative model 150 that is trained to generate new molecules given hard and soft molecule fragments as input. Techniques for training the molecular generative model 150 are discussed in greater detail below in conjunction with FIGS. 5-6. Training data and/or trained machine learning models, including the molecular generative model 150, can be stored in the data store 120, or elsewhere. In some embodiments, the data store 120 can include any storage device or devices, such as fixed disc drive(s), flash drive(s), optical storage, network attached storage (NAS), and/or a storage area-network (SAN). Although shown as accessible over the network 130, in at least one embodiment the machine learning server 110 can include the data store 120.

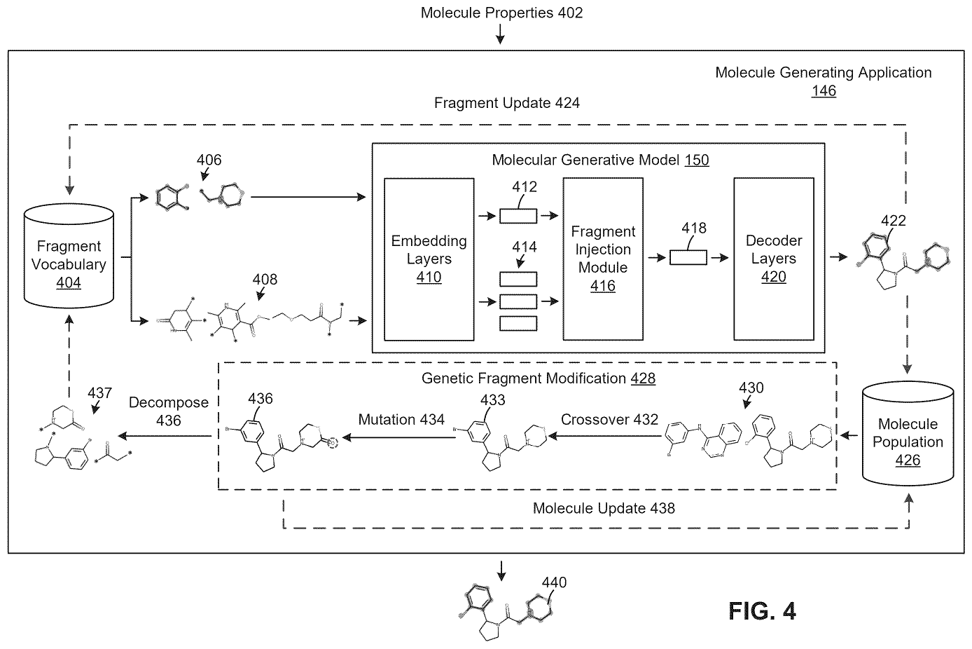

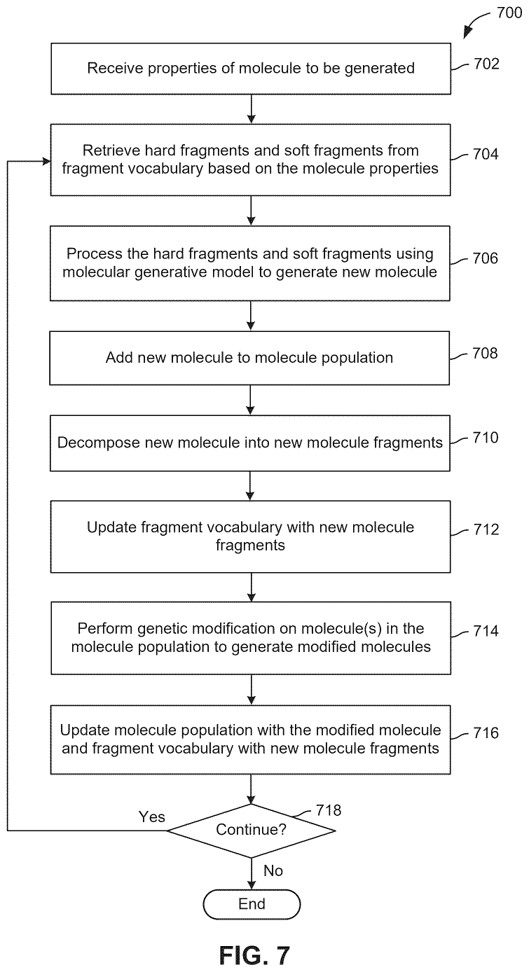

As shown, the molecule generating application 146 that uses the trained molecular generative model 150 is stored in the system memory 144, and executes on processor(s) 142, of the computing device 140. The system memory 144 and the processor(s) 142 may be similar to the system memory 114 and the processors 112, respectively, of the machine learning server, described above. The molecule generating application 146 is discussed in greater detail below in conjunction with FIGS. 4 and 7.

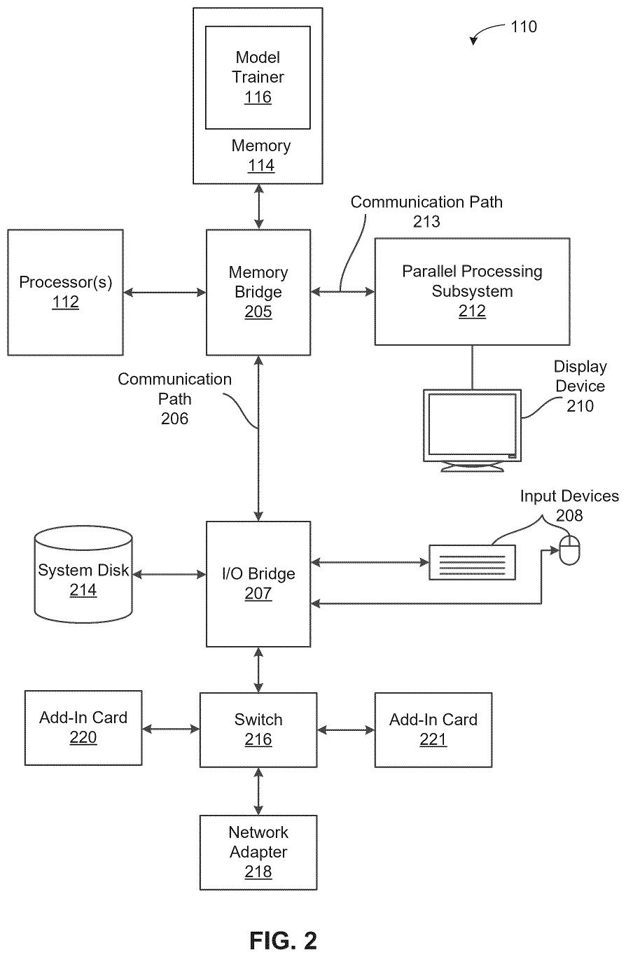

FIG. 2 is a block diagram illustrating the machine learning server 110 of FIG. 1 in greater detail, according to various embodiments. The machine learning server 110 may include any type of computing system, including, without limitation, a server machine, a server platform, a desktop machine, a laptop machine, a hand-held/mobile device, a digital kiosk, an in-vehicle infotainment system, and/or a wearable device. In some embodiments, the machine learning server 110 is a server machine operating in a data center or a cloud computing environment that provides scalable computing resources as a service over a network. In some embodiments, the machine learning server 110 can include one or more similar components as the machine learning server 110.

In various embodiments, the machine learning server 110 includes, without limitation, the processor(s) 112 and the memory(ies) 114 coupled to a parallel processing subsystem 212 via a memory bridge 205 and a communication path 213. Memory bridge 205 is further coupled to an I/O (input/output) bridge 207 via a communication path 206, and I/O bridge 207 is, in turn, coupled to a switch 216. The memory 114 stores, without limitation, the model trainer 116.

In one embodiment, I/O bridge 207 is configured to receive user input information from optional input devices 208, such as a keyboard, mouse, touch screen, sensor data analysis (e.g., evaluating gestures, speech, or other information about one or more uses in a field of view or sensory field of one or more sensors), and/or the like, and forward the input information to the processor(s) 112 for processing. In some embodiments, the machine learning server 110 may be a server machine in a cloud computing environment. In such embodiments, machine learning server 110 may not include input devices 208, but may receive equivalent input information by receiving commands (e.g., responsive to one or more inputs from a remote computing device) in the form of messages transmitted over a network and received via the network adapter 218. In some embodiments, switch 216 is configured to provide connections between I/O bridge 207 and other components of the machine learning server 110, such as a network adapter 218 and various add-in cards 220 and 221.

In some embodiments, I/O bridge 207 is coupled to a system disk 214 that may be configured to store content and applications and data for use by processor(s) 112 and parallel processing subsystem 212. In one embodiment, system disk 214 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 207 as well.

In various embodiments, memory bridge 205 may be a Northbridge chip, and I/O bridge 207 may be a Southbridge chip. In addition, communication paths 206 and 213, as well as other communication paths within machine learning server 110, may be implemented using any technically suitable protocols, including, without limitation, AGP (Accelerated Graphics Port), HyperTransport, or any other bus or point-to-point communication protocol known in the art.

In some embodiments, parallel processing subsystem 212 comprises a graphics subsystem that delivers pixels to an optional display device 210 that may be any conventional cathode ray tube, liquid crystal display, light-emitting diode display, and/or the like. In such embodiments, the parallel processing subsystem 212 may incorporate circuitry optimized for graphics and video processing, including, for example, video output circuitry. Such circuitry may be incorporated across one or more parallel processing units (PPUs), also referred to herein as parallel processors, included within the parallel processing subsystem 212.

In some embodiments, the parallel processing subsystem 212 incorporates circuitry optimized (e.g., that undergoes optimization) for general purpose and/or compute processing. Again, such circuitry may be incorporated across one or more PPUs included within parallel processing subsystem 212 that are configured to perform such general purpose and/or compute operations. In yet other embodiments, the one or more PPUs included within parallel processing subsystem 212 may be configured to perform graphics processing, general purpose processing, and/or compute processing operations. System memory 114 includes at least one device driver configured to manage the processing operations of the one or more PPUs within parallel processing subsystem 212. In addition, the system memory 114 includes the model trainer 116. Although described herein primarily with respect to the model trainer 116, techniques disclosed herein can also be implemented, either entirely or in part, in other software and/or hardware, such as in the parallel processing subsystem 212.

In various embodiments, parallel processing subsystem 212 may be integrated with one or more of the other elements of FIG. 2 to form a single system. For example, parallel processing subsystem 212 may be integrated with processor(s) 112 and other connection circuitry on a single chip to form a system on a chip (SoC).

In some embodiments, processor(s) 112 includes the primary processor of machine learning server 110, controlling and coordinating operations of other system components. In some embodiments, the processor(s) 112 issues commands that control the operation of PPUs. In some embodiments, communication path 213 is a PCI Express link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processor(s) 112, and the number of parallel processing subsystems 212, may be modified as desired. For example, in some embodiments, system memory 114 could be connected to the processor(s) 112 directly rather than through memory bridge 205, and other devices may communicate with system memory 114 via memory bridge 205 and processor(s) 112. In other embodiments, parallel processing subsystem 212 may be connected to I/O bridge 207 or directly to processor(s) 112, rather than to memory bridge 205. In still other embodiments, I/O bridge 207 and memory bridge 205 may be integrated into a single chip instead of existing as one or more discrete devices. In certain embodiments, one or more components shown in FIG. 2 may not be present. For example, switch 216 could be eliminated, and network adapter 218 and add-in cards 220, 221 would connect directly to I/O bridge 207. Lastly, in certain embodiments, one or more components shown in FIG. 2 may be implemented as virtualized resources in a virtual computing environment, such as a cloud computing environment. In particular, the parallel processing subsystem 212 may be implemented as a virtualized parallel processing subsystem in at least one embodiment. For example, the parallel processing subsystem 212 may be implemented as a virtual graphics processing unit(s) (vGPU(s)) that renders graphics on a virtual machine(s) (VM(s)) executing on a server machine(s) whose GPU(s) and other physical resources are shared across one or more VMs.

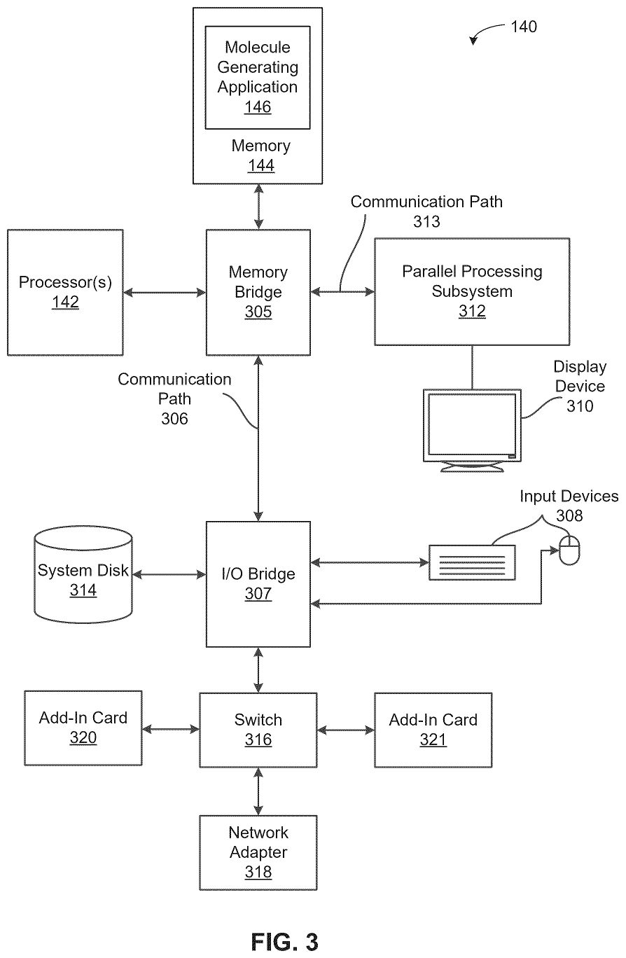

FIG. 3 is a block diagram illustrating the computing device 140 of FIG. 1 in greater detail, according to various embodiments. The computing device 140 may include any type of computing system, including, without limitation, a server machine, a server platform, a desktop machine, a laptop machine, a hand-held/mobile device, a digital kiosk, an in-vehicle infotainment system, and/or a wearable device. In some embodiments, the computing device 140 is a server machine operating in a data center or a cloud computing environment that provides scalable computing resources as a service over a network. In some embodiments, the machine learning server 110 can include one or more similar components as the computing device 140.

In various embodiments, the computing device 140 includes, without limitation, the processor(s) 142 and the memory (ies) 144 coupled to a parallel processing subsystem 312 via a memory bridge 305 and a communication path 313. Memory bridge 305 is further coupled to an I/O (input/output) bridge 307 via a communication path 306, and I/O bridge 307 is, in turn, coupled to a switch 316. The memory 144 stores, without limitation, the molecule generating application 146 that includes the molecular generative model 150.

In one embodiment, I/O bridge 307 is configured to receive user input information from optional input devices 308, such as a keyboard, mouse, touch screen, sensor data analysis (e.g., evaluating gestures, speech, or other information about one or more uses in a field of view or sensory field of one or more sensors), and/or the like, and forward the input information to the processor(s) 142 for processing. In some embodiments, the computing device 140 may be a server machine in a cloud computing environment. In such embodiments, computing device 140 may not include input devices 308, but may receive equivalent input information by receiving commands (e.g., responsive to one or more inputs from a remote computing device) in the form of messages transmitted over a network and received via the network adapter 318. In some embodiments, switch 316 is configured to provide connections between I/O bridge 307 and other components of the computing device 140, such as a network adapter 318 and various add-in cards 320 and 321.

In some embodiments, I/O bridge 307 is coupled to a system disk 314 that may be configured to store content and applications and data for use by processor(s) 142 and parallel processing subsystem 312. In one embodiment, system disk 314 provides non-volatile storage for applications and data and may include fixed or removable hard disk drives, flash memory devices, and CD-ROM (compact disc read-only-memory), DVD-ROM (digital versatile disc-ROM), Blu-ray, HD-DVD (high-definition DVD), or other magnetic, optical, or solid state storage devices. In various embodiments, other components, such as universal serial bus or other port connections, compact disc drives, digital versatile disc drives, film recording devices, and the like, may be connected to I/O bridge 307 as well.

In various embodiments, memory bridge 305 may be a Northbridge chip, and I/O bridge 307 may be a Southbridge chip. In addition, communication paths 306 and 313, as well as other communication paths within computing device 140, may be implemented using any technically suitable protocols, including, without limitation, AGP (Accelerated Graphics Port), HyperTransport, or any other bus or point-to-point communication protocol known in the art.

In some embodiments, parallel processing subsystem 312 comprises a graphics subsystem that delivers pixels to an optional display device 310 that may be any conventional cathode ray tube, liquid crystal display, light-emitting diode display, and/or the like. In such embodiments, the parallel processing subsystem 312 may incorporate circuitry optimized for graphics and video processing, including, for example, video output circuitry. Such circuitry may be incorporated across one or more parallel processing units (PPUs), also referred to herein as parallel processors, included within the parallel processing subsystem 312.

In some embodiments, the parallel processing subsystem 312 incorporates circuitry optimized (e.g., that undergoes optimization) for general purpose and/or compute processing. Again, such circuitry may be incorporated across one or more PPUs included within parallel processing subsystem 312 that are configured to perform such general purpose and/or compute operations. In yet other embodiments, the one or more PPUs included within parallel processing subsystem 312 may be configured to perform graphics processing, general purpose processing, and/or compute processing operations. System memory 144 includes at least one device driver configured to manage the processing operations of the one or more PPUs within parallel processing subsystem 312. In addition, the system memory 144 includes the speech application 146. Although described herein primarily with respect to the speech application 146, techniques disclosed herein can also be implemented, either entirely or in part, in other software and/or hardware, such as in the parallel processing subsystem 312.

In various embodiments, parallel processing subsystem 312 may be integrated with one or more of the other elements of FIG. 3 to form a single system. For example, parallel processing subsystem 312 may be integrated with processor 142 and other connection circuitry on a single chip to form a system on a chip (SoC).

In some embodiments, processor(s) 142 includes the primary processor of computing device 140, controlling and coordinating operations of other system components. In some embodiments, the processor(s) 142 issues commands that control the operation of PPUs. In some embodiments, communication path 313 is a PCI Express link, in which dedicated lanes are allocated to each PPU. Other communication paths may also be used. The PPU advantageously implements a highly parallel processing architecture, and the PPU may be provided with any amount of local parallel processing memory (PP memory).

It will be appreciated that the system shown herein is illustrative and that variations and modifications are possible. The connection topology, including the number and arrangement of bridges, the number of processor(s) 142, and the number of parallel processing subsystems 312, may be modified as desired. For example, in some embodiments, system memory 144 could be connected to the processor(s) 142 directly rather than through memory bridge 305, and other devices may communicate with system memory 144 via memory bridge 305 and processor 142. In other embodiments, parallel processing subsystem 312 may be connected to I/O bridge 307 or directly to processor 142, rather than to memory bridge 305. In still other embodiments, I/O bridge 307 and memory bridge 305 may be integrated into a single chip instead of existing as one or more discrete devices. In certain embodiments, one or more components shown in FIG. 3 may not be present. For example, switch 316 could be eliminated, and network adapter 318 and add-in cards 320, 321 would connect directly to I/O bridge 307. Lastly, in certain embodiments, one or more components shown in FIG. 3 may be implemented as virtualized resources in a virtual computing environment, such as a cloud computing environment. In particular, the parallel processing subsystem 312 may be implemented as a virtualized parallel processing subsystem in at least one embodiment. For example, the parallel processing subsystem 312 may be implemented as a virtual graphics processing unit(s) (vGPU(s)) that renders graphics on a virtual machine(s) (VM(s)) executing on a server machine(s) whose GPU(s) and other physical resources are shared across one or more VMs.

Generating Molecules with Fragment Retrieval Augmentation

FIG. 4 is a more detailed illustration of the molecule generating application 146 of FIG. 1, according to various embodiments. As shown, the molecule generating application 146 includes, without limitation, the molecular generative model 150, a fragment vocabulary 404, and a molecule population 426. The molecular generative model 150 is a machine learning model, such as an artificial neural network, that is trained to take as input hard molecule fragments and soft molecule fragments and to generate, using the soft molecule fragments as guidance, a new molecule that includes the hard molecule fragments and one or more other fragments that may have similarities with the soft molecule fragments. The fragment vocabulary 404 stores molecule fragments (also referred to herein as “fragments”). In some embodiments, the fragment vocabulary 404 can be initialized with molecule fragments from an existing molecule library, with each fragment inheriting properties from which the fragment was derived. The molecule population 426 stores molecules that can be made up of multiple molecule fragments. Each of the fragment vocabulary 404 and the molecule population 426 can be implemented in any technically feasible manner, such as using a database, a key-value store, or the like.

In operation, the molecule generating application 146 can receive desired properties of a molecule 402 to be generated. The molecule generating application 146 retrieves, from the fragment vocabulary 404, hard fragments 406 and soft fragments 408 that are most relevant to the molecule properties 402. The hard fragments 406 are molecule fragments to be included in a newly generated molecule, i.e., the hard fragments 406 are building blocks of a new molecule. The soft fragments 408 are molecule fragments used to guide the molecular generative model 150 in generating the new molecule through a trainable fragment injection module 416 of the molecular generative model 150, discussed in greater detail below. Any number of hard fragments 406 and soft fragments 408 can be retrieved in any technically feasible manner in some embodiments. For example, two hard fragments 406 and three soft fragments 408 can be retrieved in some embodiments. As described in greater detail below, in some embodiments, two hard fragments 406, such as two arms for a linker design of a molecule, or an arm and a linker for a motif extension design of a molecule, can be retrieved. In some embodiments, the molecule generating application 146 can perform a search to identify fragments stored in the fragment vocabulary 404 that are most relevant to each property in the molecule properties 402, with the relevance being indicated by a score. For example, if one of the molecule properties 402 is binding affinity to a particular protein, then the molecule generating application 146 could search for fragments in the fragment vocabulary 404 having the highest binding affinity to the particular protein. In addition, the molecule generating application 146 can normalize the scores for each property and sum the normalized scores to obtain an average score for each fragment. Then, the molecule generating application 146 can sort the fragments by their average scores and select a number of the sorted fragments as the hard fragments 406 and another number of the sorted fragments as the soft fragments 408. For example, two of the top 100 sorted fragments could be used as the hard fragments 406, and another three of the top 100 sorted fragments could be used as the soft fragments 408.

More formally, given a set of N molecules xᵢ and corresponding properties yᵢ∈[0,1] of the molecules, denoted as

$$𝒟 = {\{ ( {x}_{i} , {y}_{i} ) \}}_{i = 1}^{N} ,$$

in some embodiments, the fragment vocabulary 404 can be constructed using an arm-linker-arm slicing algorithm to decompose each molecule x into three fragments: two arms Fₐᵣₘ (i.e., fragments that have one attachment point) and one linker Fₗᵢₙₖₑᵣ (i.e., a fragment that has two attachment points). A set of arms

$${ℱ}_{arm} = {\{ {F}_{arm , j} \}}_{j = 1}^{2 N}$$

and a set of linkers

$${ℱ}_{linker} = {\{ {F}_{linker , j} \}}_{j = 1}^{N}$$

can be obtained after the arm-linker-arm slicing algorithm is applied to the molecules

$${\{ {x}_{i} \}}_{i = 1}^{N} .$$

In audition, a score can be calculated for each fragment Fⱼ∈ [symbol] ∪ [symbol] using the average property of all molecules containing Fⱼ as their substructure as follows:

$$score ( {F}_{j} ) = \frac{1}{\vert S ( {F}_{j} ) \vert} {\sum }_{( x , y ) ∈ S ( {F}_{j} )} y , \qquad ( 1 )$$

where score (Fⱼ)∈[0,1], and S(Fⱼ)={(x, y)∈ [symbol] : Fⱼ is a fragment of x}. Intuitively, the fragment score evaluates the contribution of a given fragment to a target property of the whole molecule of which the fragment is a part. From [symbol] and [symbol] , the top-N_{frag} fragments based on the score can be used to construct an arm fragment vocabulary [symbol] ⊂ [symbol] and a linker fragment vocabulary [symbol] ⊂ [symbol] , respectively.

Given the fragment vocabularies [symbol] and [symbol] in the fragment vocabulary 404 that include high-property fragments, the molecule generating application 146 can retrieve two hard fragments 406 randomly from the vocabularies. The hard fragments 406 together form a partial molecular sequence that serves as input to a pre-trained molecular language model, such as Sequential Attachment-based Fragment Embedding Generative Pre-trained Transformer (SAFE-GPT). SAFE is a noncanonical version of simplified molecular-input line-entry system (SMILES) that represents molecules as a sequence of dot-connected fragments. The order of fragments in a SAFE string does not affect the molecular identity. Using the SAFE representation, the molecule generating application 146 forces the hard fragments 406 to be included in a newly generated molecule by providing them as an input sequence to the molecule generating application 146 to complete the rest of the sequence. In some embodiments, during generation of a molecule, with a probability of 50%, the molecule generating application 146 either (1) retrieves two hard fragments from [symbol] or (2) retrieves one fragment from [symbol] and one fragment from [symbol] . In the former case, the molecule generating application 146 can perform a linker design, which generates a new fragment that links the input fragments. In the latter case, the molecule generating application 146 can first randomly select an attachment point in the retrieved linker and combine the attachment point with the retrieved arm to form a single fragment, and then perform motif extension, which generates a new fragment that completes the molecule.

In some embodiments, given two hard fragments (e.g., hard fragments 406) as input, the molecular generative model 150 generates one new fragment to complete a molecule. The generation is augmented with the information of K retrieved soft fragments (e.g., soft fragments 408), to guide the generation. Specifically, in some embodiments, if the two hard fragments are all arms, then the molecule generating application 146 can randomly retrieve soft fragments from [symbol] . If one of the hard fragments is an arm and another is a linker, the molecule generating application 146 can retrieve soft fragments from [symbol] .

The molecule generating application 146 processes the hard fragments 406 and the soft fragments 408 using the molecular generative model 150 to generate a new molecule 422. In some embodiments, the hard fragments 406 serve as a context or a prefix of the sequence passed to the molecular generative model 150, and generation of the new molecule 422 is conditioned on the hard fragments 406, which are copied into the new molecule 422. As shown, the molecular generative model 150 includes, without limitation embedding layers 410, the fragment injection module 416 that includes one or more layers for performing cross-attention, and decoder layers 420. In some embodiments, the embedding layers 410 and the decoder layers 420 can be from a language model, such as SAFE-GPT. The hard fragments 406 and the soft fragments 408 are input into the embedding layers 410, which in response outputs an input embedding 412 and soft fragment embeddings 414, respectively. The input embedding 412 and the soft fragment embeddings 414 are then input into the fragment injection module 416, which fuses the soft fragment embeddings 414 with the input embedding 412 of the hard fragments 406 and outputs an augmented embedding 418. The fragment injection module 416 allows the molecular generative model 150 to generate new fragments by referring to the information conveyed by the soft fragments 408. The augmented embedding 418 is input into the decoder layers 420, which output the new molecule 422.

More formally, using up to the L-th layer of a language model LM^{0:L} (i.e., the embedding layers 410), the embeddings of the input sequence xᵢₙₚᵤₜ and the soft fragments

$${\{ {F}_{soft , k} \}}_{k = 1}^{K}$$

can be obtained as follows:

$${h}_{input} = {LM}^{0 : L} ( {x}_{input} ) and \qquad ( 2 )$$

$${H}_{soft} = concatenate ( [ {h}_{soft}^{1} , {h}_{soft}^{2} , … , {h}_{soft}^{K} ] ) , where$$

$${h}_{soft}^{K} = {LM}^{0 : L} ( {F}_{soft , k} ) for$$

$$k = 1 , 2 , … , K .$$

Subsequently, the molecule generating application 146 can inject the embeddings of soft fragments through the fragment injection module 416. In some embodiments, the fragment injection module 416 can use cross-attention to fuse the embeddings of the input sequence and soft fragments as follows:

$$h = FI ( {h}_{input} , {H}_{soff} ) = softmax ( \frac{Query ( {h}_{input} ) \cdot Key {( {H}_{soft} )}^{T}}{\sqrt{{d}_{Key}}} ) \cdot Value ( {H}_{soft} ) , \qquad ( 3 )$$

where FI is the fragment injection module 416, Query, Key, and Value are multi-layer perceptrons (MLPs), and d_{key} is the output dimension of Key. Next, the molecule generating application 146 can generate the new molecule 422 by decoding the augmented embedding h, 418, using the later layers of the language model (i.e., the decoder layers 420) as x_{new}=LM^{L+1:L}^{_{T}}(h), where L_{T} is the total number of layers of the model. With the fragment injection module 416, the molecule generating application 146 can utilize information of the soft fragments 408 to generate novel fragments which are also likely to contribute to the molecule properties 402.

Subsequent to generating the new molecule 422, the molecule generating application 146 stores the new molecule 422 in the molecule population 426. In some embodiments, the properties of new molecules, which fragments of the new molecules inherit, can be determined by making an oracle call to one or more molecular property evaluation functions. For example, the molecular property evaluation function(s) can include a known classifier and/or predictor for predicting the properties of molecules. In addition, the molecule generating application 146 decomposes the new molecule 422 into fragments and performs a fragment update 424 in which the fragments are stored in the fragment vocabulary 404. In some embodiments, when fragments are stored in the fragment vocabulary 404, other lowest-scoring fragments with respect to the molecule properties 402 can be removed from the fragment vocabulary 404, as discussed in greater detail below. Optionally, the molecule generating application 146 can also perform genetic fragment modification 428 on molecules stored in the molecule population 426, including the new molecule 422. In some embodiments, the genetic fragment modification 428 can include (1) crossover operation(s) 432 in which parent molecules 430 that are randomly selected from the molecule population 426 are cut at random positions at ring or non-ring positions with a probability (e.g., a probability of 50%), and random fragments from the cut are combined to generate an offspring molecule 433, and (2) mutation operation(s) 434 in which bond insertion/deletion, atom insertion/deletion, bond order swapping, or atom changes are performed on the offspring molecule 433 with a predefined probability to generate a modified molecule 436. Any suitable number of genetic modification generations can be performed per cycle in some embodiments. The molecule generating application 146 stores the modified molecule 436 in the molecule population 426. In addition, the molecule generating application 146 decomposes the modified molecule 436 into fragments 437, which are stored in the fragment vocabulary 404. In some embodiments, the fragment vocabulary 404 is dynamically updated through an iterative process that scores newly generated fragments based on equation (1) and replaces fragments in the fragment vocabulary 404 with the top-N_{frag} fragments.

In some embodiments, to further enhance exploration in the chemical space, generated fragments can be enhanced with a post-hoc genetic algorithm. In some embodiments, the population P can first be initiated with the top-top-Nₘₒₗ molecules generated by the molecular generative model 150 based on the target property y. The molecule generating application 146 can then select parent molecules randomly from the population and generate offspring molecules by the crossover and mutation operations 432 and 434. The offspring molecules can have new fragments not contained in the initial fragment vocabulary 404, and the molecule generating application 146 can again update the fragment vocabularies [symbol] and [symbol] by the top-N_{frag} fragments based on the scores of equation (1). In a subsequent generation, the population P can be updated 438 with the molecules generated so far by both the molecular generative model 150 and the genetic fragment modification 428.

The foregoing process can be repeated any number of times to generate molecules and fragments that increasingly satisfy the molecule properties 402 received as input, and one or more of the generated molecules can be output by molecule generating application 146, shown as output molecule 440. That is, the molecule generating application 146 can generate desirable molecules through multiple cycles of (1) the molecular generative model 150 generation augmented with the hard fragment retrieval and the soft fragment retrieval, and (2) the genetic fragment modification. Through such an interplay of hard fragment retrieval, soft fragment retrieval, and the genetic fragment modification, the molecule generating application 146 can exploit existing chemical knowledge through the form of fragments both explicitly and implicitly, while exploring beyond initial fragments by the dynamic vocabulary update. Accordingly, the molecule generating application 146 is able to extrapolate beyond existing molecule fragments while updating the fragment vocabulary 404 with generated fragments via the iterative refinement process that is further enhanced with post-hoc genetic fragment modification, described above. As a result, the molecule generating application 146 can achieve an improved exploration-exploitation trade-off by maintaining a pool of fragments and expanding the pool of fragments with novel and high-quality fragments through a strong generative prior. Experience has shown that the molecule generating application 146 can strike a good balance between optimization performance, diversity, novelty, and synthesizability of generated molecules.

In some embodiments, assuming that SAFE-GPT is used, the molecule generating application 146 can generate molecules according to Algorithm 1:

Algorithm 1: Generation Process

```
Input: Dataset [symbol]   , fragment vocabulary size N_{frag}, molecule population size Nₘₒₗ,
number of soft fragments K, number of total generations G, number of SAFE-GPT
generations per cycle G_{SAFE-GPT}, number of genetic algorithm (GA) generations per
cycle G_{GA}
Set [symbol]  ← top- N_{frag} arms obtained from [symbol]    (Eq. (1))
Set [symbol]  ← top- −N_{frag} linkers obtained from [symbol]    (Eq. (1))
Set [symbol]  ← Ø
Set [symbol]  ← Ø
while | [symbol]  | < G do [symbol]
  Fragment retrieval-augmented SAFE-GPT generation
  for i = 1, 2, ... , N_{SAFE-GPT} do
    Randomly retrieve two hard fragments F_{hard,1}, F_{hard,2} from [symbol]  ∪ [symbol]
    $Randomly retrieve K soft fragments {\{ {F}_{soft} \}}_{k = 1}^{K} from {𝒱}_{arm} ⋃ {𝒱}_{linker}$
    $Using {F}_{hard , 1} \cdot {F}_{hard \text{.2}} as input and {\{ {F}_{soft} \}}_{k = 1}^{K} as soft fragments , run SAFE - GPT$
    to generate a molecule x
    Update [symbol]  & [symbol]  ∪ {x}
```

| | | |
|---|---|---|
| Decompose x into F_{arm,1}, Fₗᵢₙₖₑᵣ, and F_{arm,2} | | |
| Update [symbol] ← top- N_{frag} arms from [symbol] ∪ {F_{arm,1}, F_{arm,2}] | {close oversize brace} | Update |
| Update [symbol] ← top- N_{frag} linkers from [symbol] ∪ Fₗᵢₙₖₑᵣ | | |
| Update [symbol] ← top- Nₘₒₗ from [symbol] ∪ {x} | {close oversize brace} | Update |

```
  end for [symbol]
  GA generation
  for i = 1,2, ... , N_{GA} do
    Select parent molecules from [symbol]
    Perform crossover and mutation to generate a molecule x
    Update [symbol]  & [symbol]  ∪ {x}
```

| | | |
|---|---|---|
| Decompose x into F_{arm,}, Fₗᵢₙₖₑᵣ, and F_{arm,2} | | |
| Update [symbol] ← top- N_{frag} arms from [symbol] ∪ {F_{arm,1}, F_{arm,2}} | {close oversize brace} | Update |
| Update [symbol] ← top- N_{frag} linkers from [symbol] ∪ Fₗᵢₙₖₑᵣ) | | |
| Update [symbol] ← top- Nₘₒₗ from [symbol] ∪ {x} | {close oversize brace} | Update |

end for
end while
Output: Generated molecules

[symbol]

FIG. 5 is a more detailed illustration of the model trainer 116 of FIG. 1, according to various embodiments. As shown, the model trainer 116 trains the molecular generative model 150 by updating parameters of the molecular generative model 150 to generate a trained molecular generative model 150. In operation, the model trainer 116 takes as input training molecules, shown as training molecule 502 that includes fragments F₁, F₂, and F₃, from a training dataset (not shown) that includes multiple molecules. For each training molecule, the model trainer 116 retrieves, from a training pool (i.e., dataset) of fragments, multiple fragments that are most similar to a first fragment, shown as the fragment F₃, in the molecule. The most similar fragments can be determined using any technically feasible similarity metric, such as pairwise Tanimoto similarity using Morgan fingerprints of radius 2 and 1024 bits, in some embodiments. The model trainer 116 inputs (1) other fragments in the selected molecule, shown as input sequence F₁. F₂, as hard fragments and (2) the first fragment, F₃, and the multiple other fragments that are most similar to the first fragment, shown as fragments, except for a most similar fragment to the first fragment, i.e.,

$${F}_{3}^{2 NN} , {F}_{3}^{3 NN} , … , {F}_{3}^{KNN}$$

into the molecular generative model 150 to generate a new molecule 513, shown as F₁. F₂,

$${\hat{F}}_{3}^{1 NN} ,$$

which is akin to a similarity interpolation task. Although described herein primarily with respect to the most similar fragment to the first fragment as a reference example, any other fragment from the multiple other fragments that are most similar to the first fragment can be used in some embodiments.

As described above in conjunction with FIG. 4, the molecular generative model 150 includes the embedding layers 410, the fragment injection module 416, and the decoder layers 420. The input sequence 504 is input as hard fragments, and the first fragment and similar fragments, F₃,

$${F}_{3}^{2 NN} , {F}_{3}^{3 NN} , … , {F}_{3}^{KNN} ,$$

are input as soft fragments 506 into the embedding layers 410, which in response outputs an input embedding 508 and soft fragment embeddings 510, respectively. The input embedding 508 and the soft fragment embeddings 510 are then input into the fragment injection module 416, which fuses the input embedding 508 and the soft fragment embeddings 510 to generate an augmented embedding 512. The augmented embedding 512 is input into the decoder layers 420, which outputs the new molecule 513, F₁. F₂.

$${\hat{F}}_{3}^{1 NN} .$$

The model trainer 116 updates 520 parameters of the molecular generative model 150 based on a comparison between a fragment, shown as output sequence 516,

$${\hat{F}}_{3}^{1 NN} ,$$

in the new molecule 513 corresponding to the first fragment, F₃, and a most similar fragment, shown as target sequence 514,

$${F}_{3}^{1 NN} ,$$

to the first fragment, F₃. Illustratively, in some embodiments, the comparison between the fragment

$${\hat{F}}_{3}^{1 NN}$$

from the new molecule 513 and the most similar fragment

$${F}_{3}^{1 NN}$$

can be computed as a cross entropy 518 between the fragment

$${\hat{F}}_{3}^{1 NN}$$

from the new molecule 513 and the most similar fragment

$${F}_{3}^{1 NN} .$$

In such cases, the model trainer 116 can update parameters of the molecular generative model 150 using the cross entropy 518 as a loss function and backpropagation with gradient descent, or a variant thereof. In some embodiments, the molecular generative model 150 can include embedding layers 410 and decoder layers 420 from a pre-trained model, such as SAFE-GPT. In such cases, the model trainer 116 can keep parameters of the embedding layers 410 and decoder layers 420 fixed while updating parameters of the fragment injection module 416.

The foregoing training process can be repeated for any number of iterations, using a new training molecule to update the molecular generative model 150 at each iteration. For example, in some embodiments, training can continue for a predefined number of iterations, until the loss (e.g., the cross entropy 518) plateaus, or the like.

More formally, in some embodiments, the model trainer 116 uses a self-supervised objective that predicts the most similar fragment to the input fragments. Specifically, each molecular sequence x in the training set can be first decomposed into fragment sequences (F₁, F₂, F₃) with a random permutation between the fragments, using the same slicing algorithm used in the construction of the fragment vocabulary 404, described above in conjunction with FIG. 4. A molecule x can be represented by connecting fragments of the molecule with dots as, for example, F₁·F₂·F₃. During training, the model trainer 116 can consider a number of the fragments, such as the first two fragments as hard fragments. Given the remaining fragment F₃, the model trainer 116 retrieves K most similar fragments

$${\{ {F}_{3}^{kNN} \}}_{k = 1}^{K}$$

from a training fragment pool. As described, the pairwise Tanimoto similarity using Morgan fingerprints of radius 2 and 1024 bits can be employed to determine the most similar fragments in some embodiments. Using the hard fragments as the input sequence as F₁. F₂, the objective can used be to predict the most similar fragment

$${F}_{3}^{1 NN}$$

utilizing the original fragment and the next K−1 most similar fragments

$${\{ {F}_{3}^{kNN} \}}_{k = 2}^{K}$$

as the soft fragments. It should be noted that the training can be target property-agnostic, as the fragments used for training are independent of the target property. By contrast, the fragment vocabulary 404 used for generating molecules can be target property-specific, as the fragment vocabulary 404 is constructed using the scoring function of equation (1). Accordingly, the molecule generating application 146 can effectively generate optimized molecules across different target properties without any retraining of the molecular generative model 150.

FIG. 6 is a flow diagram of method steps for training a molecular generative model, according to various embodiments. Although the method steps are described in conjunction with the systems of FIGS. 1-5, persons skilled in the art will understand that any system configured to perform the method steps in any order falls within the scope of the present embodiments.

As shown, a method 600 begins at step 602, where the model trainer 116 selects a molecule to use for training. The molecule can be selected from a training dataset of molecules.

At step 604, the model trainer 116 retrieves multiple fragments that are most similar to a first fragment in the selected molecule. In some embodiments, the most similar fragments are retrieved from a training pool of fragments, and the most similar fragments can be determined using any technically feasible similarity metric. For example, in some embodiments, the pairwise Tanimoto similarity using Morgan fingerprints of radius 2 and 1024 bits can be used.

At step 606, the model trainer 116 inputs (1) other fragments in the selected molecule as hard fragments and (2) the first fragment and the multiple other fragments that are most similar to the first fragment, except for a most similar fragment to the first fragment, into the molecular generative model 150 being trained. Given such inputs, the molecular generative model 150 outputs a new molecule.

At step 608, the model trainer 116 updates parameters of the molecular generative model 150 based on a comparison between a fragment in the new molecule corresponding to the first fragment and the most similar fragment to the first fragment. The molecular generative model 150 can be updated in any technically feasible manner in some embodiments. In some embodiments, the comparison between the fragment in the new molecule corresponding to the first fragment and the most similar fragment to the first fragment can be computed as a cross entropy. In such cases, the model trainer 116 can update parameters of the molecular generative model 150 using the cross entropy as a loss function and backpropagation with gradient descent, or a variant thereof. In some embodiments, the molecular generative model 150 can include embedding layers 410 and decoder layers 420 from a pre-trained model, such as SAFE-GPT. In such cases, the model trainer 116 can keep parameters of the embedding layers 410 and decoder layers 420 fixed while updating parameters of the fragment injection module 416.

At step 610, if training is to continue, then the method 600 returns to step 602, where the model trainer 116 selects another molecule to use for training. In some embodiments, training can continue for a predefined number of iterations, until a loss (e.g., the cross entropy 518) plateaus, or the like. On the other hand, if training is not to continue, then the method 600 ends.

FIG. 7 is a flow diagram of method steps for generating molecules using a trained molecular generative model, according to various embodiments. Although the method steps are described in conjunction with the systems of FIGS. 1-5, persons skilled in the art will understand that any system configured to perform the method steps in any order falls within the scope of the present embodiments.

As shown, a method 700 begins at step 702, where the molecule generating application 146 receives properties of a molecule to be generated. A user can input desired molecule properties in any technically feasible manner, such as via a user interface (UI), in some embodiments.

At step 704, the molecule generating application 146 retrieves hard fragments and soft fragments from the fragment vocabulary 404 based on the received molecule properties. As described, any number of hard fragments and soft fragments can be retrieved in any technically feasible manner in some embodiments. In some embodiments, two hard fragments 406, such as two arms for a linker design of a molecule, or an arm and a linker for a motif extension design of a molecule, can be retrieved. In some embodiments, the molecule generating application 146 can perform a search to identify fragments stored in the fragment vocabulary 404 that are most relevant to each property in the molecule properties 402, with the relevance being indicated by a score. In addition, the molecule generating application 146 can normalize the scores for the properties and sum the normalized scores to obtain an average score for each fragment. Then, the molecule generating application 146 can sort the fragments by their average scores and select a number (e.g., 2) of the sorted fragments as the hard fragments and another number (e.g., 3) of the sorted fragments as the soft fragments, as described above in conjunction with FIG. 4.

At step 706, the molecule generating application 146 processes the hard fragments and the soft fragments using the molecular generative model 150 to generate a new molecule. As described above in conjunction with FIG. 4, the molecular generative model 150 includes the embedding layers 410, the fragment injection module 416, and the decoder layers 420. Given the hard fragments and soft fragments as inputs, the embedding layers 410 generates an input embedding and soft fragment embeddings, respectively. In turn, the input embedding and the soft fragment embeddings are input into the fragment injection module 416, which fuses (i.e., mixes) the input embedding and the soft fragment embeddings to generate an augmented embedding. Then, the augmented embedding is input into the decoder layers 420, which outputs the new molecule.

At step 708, the molecule generating application 146 adds the new molecule to the molecule population 426. As described, in some embodiments, the properties of the new molecule, which fragments of the new molecules inherit, can be determined by making an oracle call to one or more molecular property evaluation functions. Any technically feasible oracle call, such as a call to a known classifier and/or predictor for predicting the properties of molecules, can be made in some embodiments.

At step 710, the molecule generating application 146 decomposes the new molecule into new molecule fragments, and, at step 712, the molecule generating application 146 updates the fragment vocabulary 404 with the new molecule fragments. In some embodiments, when fragments are stored in the fragment vocabulary 404, other lowest-scoring fragments with respect to the molecule properties received at step 702 can be removed from the fragment vocabulary 404.

At step 714, the molecule generating application 146 performs genetic modification on one or more molecules in the molecule population to generate modified molecules. Steps 714-716 are optional and may not be performed in some embodiments. In some embodiments, the genetic modification can include (1) crossover operation(s) in which parent molecules that are randomly selected from the molecule population 426 are cut at random positions at ring or non-ring positions with a probability (e.g., a probability of 50%), and random fragments from the cut are combined to generate an offspring molecule, and (2) mutation operation(s) in which bond insertion/deletion, atom insertion/deletion, bond order swapping, or atom changes are performed on the offspring molecule with a predefined probability to generate the modified molecule. Any suitable number of genetic modification generations can be performed at step 714 in some embodiments.

At step 716, the molecule generating application 146 updates the molecule population 426 with the modified molecule and the fragment vocabulary 404 with the new molecule fragments from the modified molecule. In some embodiments, the molecule generating application 146 decomposes the modified molecule into fragments and stores the fragments in the fragment vocabulary 404.

At step 718, if the molecule generating application 146 determines to continue generating molecules, then the method 700 returns to step 704, where the molecule generating application 146 again retrieves hard fragments and soft fragments from the fragment vocabulary 404 based on the molecule properties. The molecule generating application 146 can determine whether to continue based on any suitable stopping condition in some embodiments. For example, in some embodiments, the stopping condition can depend on a budget on the number of oracle calls that can be made (i.e., how many assessments of the generated molecules a user can afford by calling molecular property evaluation functions).

By repeating the steps 704-718, molecules and fragments can be generated that increasingly satisfy the molecule properties received as input at step 702.

In sum, techniques are disclosed for generating molecules using fragment retrieval augmentation. In some embodiments, a molecule generating application takes as input desired properties of a molecule. The molecule generating application retrieves, from a fragment vocabulary, a number of hard fragments that a newly generated molecule must include and a number of soft fragments that guide the generation of the new molecule. The molecule generating application processes the hard fragments and the soft fragments using a trained molecular generative model to generate a new molecule. The molecule generating application adds the new molecule to a molecule population. The molecule generating application also decomposes the new molecule into new molecule fragments that are added to the fragment vocabulary. Optionally, the molecule generating application performs genetic modification, such as crossover and mutation operations, using molecules in the molecule population to generate modified molecules, which can be added to the molecule population and decomposed into molecule fragments that are added to the fragment vocabulary. The foregoing process can be repeated any number of times to generate molecules and fragments that increasingly satisfy the desired molecule properties received as input.

To train the molecular generative model, a model trainer uses a number of molecules from a training dataset. For each molecule selected from the training dataset, the model trainer retrieves multiple fragments that are most similar to a first fragment in the selected molecule. The model trainer inputs (1) other fragments in the selected molecule as hard fragments, and (2) the first fragment and the multiple other fragments that are most similar to the first fragment, except for a most similar fragment to the first fragment, into the molecular generative model being trained. Given such inputs, the molecular generative model outputs a new molecule. Then, the model trainer updates parameters of the molecular generative model based on a comparison, such as a cross-entropy loss, between a fragment in the new molecule corresponding to the first fragment and the most similar fragment to the first fragment.

At least one technical advantage of the disclosed techniques relative to the prior art is that, with the disclosed techniques, molecules are generated that include, but are not limited to, known molecule fragments. In some cases, the generated molecules can exhibit a set of desired properties to a higher degree than molecules that are generated by simply combining known molecule fragments. That is, a broader range of molecules can be generated using the disclosed techniques, increasing the likelihood of generating molecules with improved properties over prior art approaches. Further, molecules that are generated according to the disclosed techniques can generally be synthesized in real life. These technical advantages represent one or more technological improvements over prior art approaches.

1. In some embodiments, a computer-implemented method for generating molecules comprises selecting, based on one or more molecule properties, one or more hard molecule fragments and one or more soft molecule fragments, and processing, using a trained machine learning model, the one or more hard molecule fragments and the one or more soft molecule fragments to generate a molecule, wherein the molecule includes the one or more hard molecule fragments, and wherein the trained machine learning model generates the molecule based on the one or more soft molecule fragments.

2. The computer-implemented method of clause 1, further comprising performing one or more genetic modifications using the molecule to generate a modified molecule.

3. The computer-implemented method of clauses 1 or 2, wherein the one or more genetic modifications comprise at least one of a crossover operation or a mutation operation.

4. The computer-implemented method of any of clauses 1-3, further comprising storing, in a fragment vocabulary, a plurality of molecule fragments included in the modified molecule.

5. The computer-implemented method of any of clauses 1-4, wherein selecting the one or more hard molecule fragments and the one or more soft molecule fragments comprises retrieving a plurality of molecule fragments from a fragment vocabulary based on the one or more molecule properties, and selecting the one or more hard molecule fragments and the one or more soft molecule fragments from the plurality of molecule fragments.

6. The computer-implemented method of any of clauses 1-5, further comprising decomposing the molecule into another plurality of molecule fragments, and storing the another plurality of molecule fragments in the fragment vocabulary.

7. The computer-implemented method of any of clauses 1-6, wherein the trained machine learning model comprises one or more embedding layers that generate one or more first embeddings based on the one or more hard molecule fragments and the one or more soft molecule fragments, one or more cross-attention layers that generate a second embedding based on the one or more first embeddings, and one or more decoder layers that generate the molecule based on the second embedding.

8. The computer-implemented method of any of clauses 1-7, further comprising storing the molecule in a molecule population that stores a plurality of different molecules.

9. The computer-implemented method of any of clauses 1-8, further comprising storing, in a fragment vocabulary, a plurality of molecule fragments included in the molecule, selecting, from the fragment vocabulary and based on the one or more molecule properties, one or more additional hard molecule fragments and one or more additional soft molecule fragments, and processing, using the trained machine learning model, the one or more additional hard molecule fragments and the one or more additional soft molecule fragments to generate another molecule.

10. The computer-implemented method of any of clauses 1-9, wherein each hard molecule fragment included in the one or more hard molecule fragments comprises a linker or an arm.

11. In some embodiments, one or more non-transitory computer-readable media store instructions that, when executed by at least one processor, cause the at least one processor to perform the steps of selecting, based on one or more molecule properties, one or more hard molecule fragments and one or more soft molecule fragments, and processing, using a trained machine learning model, the one or more hard molecule fragments and the one or more soft molecule fragments to generate a molecule, wherein the molecule includes the one or more hard molecule fragments, and wherein the trained machine learning model generates the molecule based on the one or more soft molecule fragments.

12. The one or more non-transitory computer-readable media of clause 11, wherein the instructions, when executed by the at least one processor, further cause the at least one processor to perform the step of performing one or more genetic modifications using the molecule to generate a modified molecule.

13. The one or more non-transitory computer-readable media of clauses 11 or 12, wherein selecting the one or more hard molecule fragments and the one or more soft molecule fragments comprises retrieving a plurality of molecule fragments from a fragment vocabulary based on the one or more molecule properties, and selecting the one or more hard molecule fragments and the one or more soft molecule fragments from the plurality of molecule fragments.

14. The one or more non-transitory computer-readable media of any of clauses 11-13, wherein the instructions, when executed by the at least one processor, further cause the at least one processor to perform the steps of decomposing the molecule into another plurality of molecule fragments, and storing the another plurality of molecule fragments in the fragment vocabulary.

15. The one or more non-transitory computer-readable media of any of clauses 11-14, wherein the trained machine learning model is configured to receive as input one or more hard fragments and one or more soft fragments and to generate an output molecule.

16. The one or more non-transitory computer-readable media of any of clauses 11-15, wherein the trained machine learning model comprises one or more embedding layers that generate one or more first embeddings based on the one or more hard molecule fragments and the one or more soft molecule fragments, one or more cross-attention layers that generate a second embedding based on the one or more first embeddings, and one or more decoder layers that generate the molecule based on the second embedding.

17. The one or more non-transitory computer-readable media of any of clauses 11-16, wherein the instructions, when executed by the at least one processor, further cause the at least one processor to perform the steps of storing, in a fragment vocabulary, a plurality of molecule fragments included in the molecule, selecting, from the fragment vocabulary and based on the one or more molecule properties, one or more additional hard molecule fragments and one or more additional soft molecule fragments, and processing, using the trained machine learning model, the one or more additional hard molecule fragments and the one or more additional soft molecule fragments to generate another molecule.

18. The one or more non-transitory computer-readable media of any of clauses 11-17, wherein the one or more hard molecule fragments includes two arms.

19. The one or more non-transitory computer-readable media of any of clauses 11-18, wherein the one or more hard molecule fragments include a linker and an arm.

20. In some embodiments, a system comprises one or more memories storing instructions, and one or more processors that are coupled to the one or more memories and, when executing the instructions, are configured to select, based on one or more molecule properties, one or more hard molecule fragments and one or more soft molecule fragments, and process, using a trained machine learning model, the one or more hard molecule fragments and the one or more soft molecule fragments to generate a molecule, wherein the molecule includes the one or more hard molecule fragments, and wherein the trained machine learning model generates the molecule based on the one or more soft molecule fragments.

1. In some embodiments, a computer-implemented method for training a machine learning model to generate molecules comprises selecting a plurality of molecule fragments that are most similar to a first molecule fragment included in a first molecule, processing, using an untrained machine learning model, one or more other molecule fragments included in the first molecule, the first molecule fragment, and the plurality of molecule fragments except for a second molecule fragment included in the plurality of molecule fragments to generate a second molecule, and updating, based on a comparison between a third molecule fragment included in the second molecule and the second molecule fragment, one or more parameters of the untrained machine learning model to generate a trained machine learning model.

2. The computer-implemented method of clause 1, wherein the one or more other molecule fragments are input into the untrained machine learning model as one or more hard molecule fragments that need to be included in the second molecule.

3. The computer-implemented method of clauses 1 or 2, wherein the plurality of molecule fragments except for the second molecule fragment are input into the untrained machine learning model as a plurality of soft molecule fragments that are used to guide generation of the second molecule.

4. The computer-implemented method of any of clauses 1-3, wherein the comparison between the third molecule fragment and the second molecule fragment comprises computing a cross-entropy loss between the third molecule fragment and the second molecule fragment.

5. The computer-implemented method of any of clauses 1-4, wherein the plurality of molecule fragments are selected using a pairwise Tanimoto similarity metric.

6. The computer-implemented method of any of clauses 1-5, wherein the trained machine learning model comprises one or more embedding layers that generate one or more first embeddings based on one or more hard molecule fragments and one or more soft molecule fragments, one or more cross-attention layers that generate a second embedding based on the one or more first embeddings, and one or more decoder layers that generate an output molecule based on the second embedding.

7. The computer-implemented method of any of clauses 1-6, wherein the trained machine learning model is configured to receive as input one or more hard fragments and one or more soft fragments and to generate an output molecule.

8. The computer-implemented method of any of clauses 1-7, wherein selecting the plurality of molecule fragments comprises searching a dataset of molecule fragments to identify the plurality of molecule fragments.

9. The computer-implemented method of any of clauses 1-8, further comprising selecting another plurality of molecule fragments that are most similar to a fourth molecule fragment included in a third molecule, processing, using the untrained machine learning model, one or more other molecule fragments included in the third molecule, the third molecule fragment, and the another plurality of molecule fragments except for a fifth molecule fragment included in the another plurality of molecule fragments to generate a fourth molecule, and updating, based on a comparison between a sixth molecule fragment included in the fourth molecule and the fifth molecule fragment, the one or more parameters of the untrained machine learning model.

10. The computer-implemented method of any of clauses 1-9, further comprising selecting, based on one or more molecule properties, one or more hard molecule fragments and one or more soft molecule fragments, and processing, using the trained machine learning model, the one or more hard molecule fragments and the one or more soft molecule fragments to generate a third molecule.

11. In some embodiments, one or more non-transitory computer-readable media store instructions that, when executed by at least one processor, cause the at least one processor to perform the steps of selecting a plurality of molecule fragments that are most similar to a first molecule fragment included in a first molecule, processing, using an untrained machine learning model, one or more other molecule fragments included in the first molecule, the first molecule fragment, and the plurality of molecule fragments except for a second molecule fragment included in the plurality of molecule fragments to generate a second molecule, and updating, based on a comparison between a third molecule fragment included in the second molecule and the second molecule fragment, one or more parameters of the untrained machine learning model to generate a trained machine learning model.

12. The one or more non-transitory computer-readable media of clause 11, wherein the one or more other molecule fragments are input into the untrained machine learning model as one or more hard molecule fragments that need to be included in the second molecule, and wherein the plurality of molecule fragments except for the second molecule fragment are input into the untrained machine learning model as a plurality of soft molecule fragments that are used to guide generation of the second molecule.

13. The one or more non-transitory computer-readable media of clauses 11 or 12, wherein the second molecule fragment is most similar to the first molecule fragment among the plurality of molecule fragments.

14. The one or more non-transitory computer-readable media of any of clauses 11-13, wherein the trained machine learning model comprises a trained language model.

15. The one or more non-transitory computer-readable media of any of clauses 11-14, wherein the trained machine learning model further comprises one or more cross-attention layers between one or more embedding layers and one or more decoder layers of the trained language model.

16. The one or more non-transitory computer-readable media of any of clauses 11-15, wherein the trained machine learning model is configured to receive as input one or more hard fragments and one or more soft fragments and to generate an output molecule.

17. The one or more non-transitory computer-readable media of any of clauses 11-16, wherein the trained machine learning model comprises one or more embedding layers that generate one or more first embeddings based on the one or more hard molecule fragments and the one or more soft molecule fragments, one or more cross-attention layers that generate a second embedding based on the one or more first embeddings, and one or more decoder layers that generate an output molecule based on the second embedding.

18. The one or more non-transitory computer-readable media of any of clauses 11-17, wherein selecting the plurality of molecule fragments comprises searching a dataset of molecule fragments to identify the plurality of molecule fragments.

19. The one or more non-transitory computer-readable media of any of clauses 11-18, wherein the instructions, when executed by the at least one processor, further cause the at least one processor to perform the steps of selecting, based on one or more molecule properties, one or more hard molecule fragments and one or more soft molecule fragments, and processing, using the trained machine learning model, the one or more hard molecule fragments and the one or more soft molecule fragments to generate a third molecule.

20. In some embodiments, a system comprises one or more memories storing instructions, and one or more processors that are coupled to the one or more memories and, when executing the instructions, are configured to select a plurality of molecule fragments that are most similar to a first molecule fragment included in a first molecule, process, using an untrained machine learning model, one or more other molecule fragments included in the first molecule, the first molecule fragment, and the plurality of molecule fragments except for a second molecule fragment included in the plurality of molecule fragments to generate a second molecule, and update, based on a comparison between a third molecule fragment included in the second molecule and the second molecule fragment, one or more parameters of the untrained machine learning model to generate a trained machine learning model.

Any and all combinations of any of the claim elements recited in any of the claims and/or any elements described in this application, in any fashion, fall within the contemplated scope of the present disclosure and protection.

The descriptions of the various embodiments have been presented for purposes of illustration, but are not intended to be exhaustive or limited to the embodiments disclosed. Many modifications and variations will be apparent to those of ordinary skill in the art without departing from the scope and spirit of the described embodiments.

Aspects of the present embodiments may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “module” or “system.” Furthermore, aspects of the present disclosure may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

Aspects of the present disclosure are described above with reference to flowchart illustrations and/or block diagrams of methods, apparatus (systems) and computer program products according to embodiments of the disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine. The instructions, when executed via the processor of the computer or other programmable data processing apparatus, enable the implementation of the functions/acts specified in the flowchart and/or block diagram block or blocks. Such processors may be, without limitation, general purpose processors, special-purpose processors, application-specific processors, or field-programmable gate arrays.

The flowchart and block diagrams in the figures illustrate the architecture, functionality, and operation of possible implementations of systems, methods and computer program products according to various embodiments of the present disclosure. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

While the preceding is directed to embodiments of the present disclosure, other and further embodiments of the disclosure may be devised without departing from the basic scope thereof, and the scope thereof is determined by the claims that follow.

What is claimed is:

1. A computer-implemented method for training a machine learning model to generate molecules, the method comprising:
selecting a plurality of molecule fragments that are most similar to a first molecule fragment included in a first molecule;
processing, using an untrained machine learning model, one or more other molecule fragments included in the first molecule, the first molecule fragment, and the plurality of molecule fragments except for a second molecule fragment included in the plurality of molecule fragments to generate a second molecule; and
updating, based on a comparison between a third molecule fragment included in the second molecule and the second molecule fragment, one or more parameters of the untrained machine learning model to generate a trained machine learning model.

2. The computer-implemented method of claim 1, wherein the one or more other molecule fragments are input into the untrained machine learning model as one or more hard molecule fragments that need to be included in the second molecule.

3. The computer-implemented method of claim 1, wherein the plurality of molecule fragments except for the second molecule fragment are input into the untrained machine learning model as a plurality of soft molecule fragments that are used to guide generation of the second molecule.

4. The computer-implemented method of claim 1, wherein the comparison between the third molecule fragment and the second molecule fragment comprises computing a cross-entropy loss between the third molecule fragment and the second molecule fragment.

5. The computer-implemented method of claim 1, wherein the plurality of molecule fragments are selected using a pairwise Tanimoto similarity metric.

6. The computer-implemented method of claim 1, wherein the trained machine learning model comprises:
one or more embedding layers that generate one or more first embeddings based on one or more hard molecule fragments and one or more soft molecule fragments;
one or more cross-attention layers that generate a second embedding based on the one or more first embeddings; and
one or more decoder layers that generate an output molecule based on the second embedding.

7. The computer-implemented method of claim 1, wherein the trained machine learning model is configured to receive as input one or more hard fragments and one or more soft fragments and to generate an output molecule.

8. The computer-implemented method of claim 1, wherein selecting the plurality of molecule fragments comprises searching a dataset of molecule fragments to identify the plurality of molecule fragments.

9. The computer-implemented method of claim 1, further comprising:
selecting another plurality of molecule fragments that are most similar to a fourth molecule fragment included in a third molecule;
processing, using the untrained machine learning model, one or more other molecule fragments included in the third molecule, the third molecule fragment, and the another plurality of molecule fragments except for a fifth molecule fragment included in the another plurality of molecule fragments to generate a fourth molecule; and
updating, based on a comparison between a sixth molecule fragment included in the fourth molecule and the fifth molecule fragment, the one or more parameters of the untrained machine learning model.

10. The computer-implemented method of claim 1, further comprising:
selecting, based on one or more molecule properties, one or more hard molecule fragments and one or more soft molecule fragments; and
processing, using the trained machine learning model, the one or more hard molecule fragments and the one or more soft molecule fragments to generate a third molecule.

11. One or more non-transitory computer-readable media storing instructions that, when executed by at least one processor, cause the at least one processor to perform the steps of:
selecting a plurality of molecule fragments that are most similar to a first molecule fragment included in a first molecule;
processing, using an untrained machine learning model, one or more other molecule fragments included in the first molecule, the first molecule fragment, and the plurality of molecule fragments except for a second molecule fragment included in the plurality of molecule fragments to generate a second molecule; and
updating, based on a comparison between a third molecule fragment included in the second molecule and the second molecule fragment, one or more parameters of the untrained machine learning model to generate a trained machine learning model.

12. The one or more non-transitory computer-readable media of claim 11, wherein the one or more other molecule fragments are input into the untrained machine learning model as one or more hard molecule fragments that need to be included in the second molecule, and wherein the plurality of molecule fragments except for the second molecule fragment are input into the untrained machine learning model as a plurality of soft molecule fragments that are used to guide generation of the second molecule.

13. The one or more non-transitory computer-readable media of claim 11, wherein the second molecule fragment is most similar to the first molecule fragment among the plurality of molecule fragments.

14. The one or more non-transitory computer-readable media of claim 11, wherein the trained machine learning model comprises a trained language model.

15. The one or more non-transitory computer-readable media of claim 14, wherein the trained machine learning model further comprises one or more cross-attention layers between one or more embedding layers and one or more decoder layers of the trained language model.

16. The one or more non-transitory computer-readable media of claim 11, wherein the trained machine learning model is configured to receive as input one or more hard fragments and one or more soft fragments and to generate an output molecule.

17. The one or more non-transitory computer-readable media of claim 16, wherein the trained machine learning model comprises:
one or more embedding layers that generate one or more first embeddings based on the one or more hard molecule fragments and the one or more soft molecule fragments;
one or more cross-attention layers that generate a second embedding based on the one or more first embeddings; and
one or more decoder layers that generate an output molecule based on the second embedding.

18. The one or more non-transitory computer-readable media of claim 11, wherein selecting the plurality of molecule fragments comprises searching a dataset of molecule fragments to identify the plurality of molecule fragments.

19. The one or more non-transitory computer-readable media of claim 11, wherein the instructions, when executed by the at least one processor, further cause the at least one processor to perform the steps of:
selecting, based on one or more molecule properties, one or more hard molecule fragments and one or more soft molecule fragments; and
processing, using the trained machine learning model, the one or more hard molecule fragments and the one or more soft molecule fragments to generate a third molecule.

20. A system, comprising:
one or more memories storing instructions; and
one or more processors that are coupled to the one or more memories and, when executing the instructions, are configured to:
select a plurality of molecule fragments that are most similar to a first molecule fragment included in a first molecule,
process, using an untrained machine learning model, one or more other molecule fragments included in the first molecule, the first molecule fragment, and the plurality of molecule fragments except for a second molecule fragment included in the plurality of molecule fragments to generate a second molecule, and
update, based on a comparison between a third molecule fragment included in the second molecule and the second molecule fragment, one or more parameters of the untrained machine learning model to generate a trained machine learning model.
